(19) United States
(12) Patent Application Publication
(10) Pub. No.: US 2008/0255555 A1
(43) Pub. Date: Oct. 16, 2008

(54) Temporary anchorable tether systems and methods

(76) Inventors: Jeff R. Justis, Collierville, TN (US); Damian Jelich, Cottage Grove, MN (US); Eric Alan Meyer, Andover, MN (US)

Correspondence Address: Medtronic Spinal and Biologics;Attn: Noreen Johnson - IP Legal Department, 2600 Sofamor Danek Drive, Memphis, TN, 38132, US

(21) Appl. No.: 11/786,285

(22) Filed: Apr. 11, 2007

(51) Int. Cl. A61B 17/90

(52) U.S. Cl. 606/60

(57) ABSTRACT

A surgical apparatus for guiding the position of a medical device to a desired location includes an outer sleeve, a tether, and at least one elastic anchor hook engaged with the tether. The tether may be slideably moveable within the outer sleeve. Additionally, the anchor hook is configured to anchor into bone at the desired location. In certain embodiments, the anchor hook is elastically deformable from an unstressed curved shape to a deformed straightened shape. The anchor hook may be deformed to the straightened shape when positioned within the outer sleeve and may reform to the curved shape upon deployment from the outer sleeve. In certain embodiments, a rod surrounds part of the tether and is used for threading through medical devices to advance the medical devices to the desired location. Additionally, a deployment button may be used to deploy the anchor hooks from the outer sleeve.

The present disclosure broadly concerns tether systems and relates to a system involving a temporary anchorable tether system and method for introducing a medical device through a surgical opening in a body and guiding the position of the device to a desired location. The present disclosure more specifically, but not exclusively, concerns a flexible tether engaged with two elastic anchor members configured to be deployed and anchor into tissue or bone. The system can be useful in orthopedic procedures, and more specifically in procedures correcting spinal injuries or deformities.

In the realm of orthopedic surgery, it is well known to use implants to fix the position of bones. In this way, the healing of a broken bone can be promoted, and malformations or other injuries can be corrected. For example, in the field of spinal surgery, it is well known to place such implants into vertebrae for a number of reasons, including (a) correcting an abnormal curvature of the spine, including a scoliotic curvature, (b) to maintain appropriate spacing and provide support to broken or otherwise injured vertebrae, and (c) perform other therapies on the spinal column.

Additionally, numerous medical procedures have come into common usage for accessing a desired location within the body in a minimally invasive manner to perform a wide variety of diagnostic and therapeutic procedures. Such medical procedures generally involve the use of a medical instrument, such as a small diameter probe as an example, to create a pathway through the skin and tissue to the desired site. In some approaches, a guidewire is then utilized to extend through the skin to the accessed site to enable over-the-wire advancement of therapeutic, diagnostic or surgical devices, including implants or instruments, to the desired location.

It is generally well known to use guidewire or similar systems to advance the implants, or other such medical devices including instruments, to the desired location in bone or tissue. A multitude of systems exist for guiding medical devices to desired locations; however, the systems can be difficult to assemble and secure, and can cause tissue irritation and/or damage to the surrounding area. Therefore, a need exists for improved systems and methods in this area.

### BRIEF DESCRIPTION OF THE DRAWINGS

FIG. 1 is a perspective view of a temporary anchorable tether system according to one embodiment of the present disclosure.

FIG. 2 is another perspective view of a temporary anchorable tether system according to the embodiment of FIG. 1.

FIG. 3 is a side view of components of a temporary anchorable tether system according to one embodiment of FIG. 1.

FIG. 3A is a side view of components of a temporary anchorable tether system according to one embodiment of FIG. 1.

FIG. 3B is a side view of components of a temporary anchorable tether system according to one embodiment of FIG. 1.

FIG. 4 is a side view of components of a temporary anchorable tether system according to one embodiment of FIG. 1.

FIG. 5 is a side view of components of a temporary anchorable tether system according to one embodiment of FIG. 1.

FIG. 6 is a side view of components of a temporary anchorable tether system according to one embodiment of FIG. 1.

FIG. 7 is a side view of components of a temporary anchorable tether system according to one embodiment of FIG. 1.

FIG. 8 is a side view of components of a temporary anchorable tether system according to one embodiment of FIG. 1.

FIG. 9 is a part cross-sectional side view of an embodiment of a temporary anchorable tether system.

### DESCRIPTION OF THE ILLUSTRATED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated devices, and such further applications of the principles of the disclosure as illustrated therein, being contemplated as would normally occur to one skilled in the art to which the disclosure relates.

The present disclosure provides for temporary anchorable tether systems and methods. The systems can include tethers with retaining, elastic anchor members to be placed through holes created in the body by a surgical instrument. The anchor members of the systems provide a substantially blunt profile to the accessed tissue or bone and reduce the likelihood of damage caused by undesired advancement of the system into the tissue or bone. In certain embodiments, the anchor members are composed of a shape memory alloy and are thereby deformable to be received in an outer sleeve and reform to a curved configuration upon deployment from the outer sleeve to anchor into tissue or bone at a desired location. The systems can include a push rod or needle associated with the tether around which various medical devices can be placed, such as instruments and/or implants, to position the devices adjacent the desired location. Methods of assembling and utilizing the systems described herein are also provided.

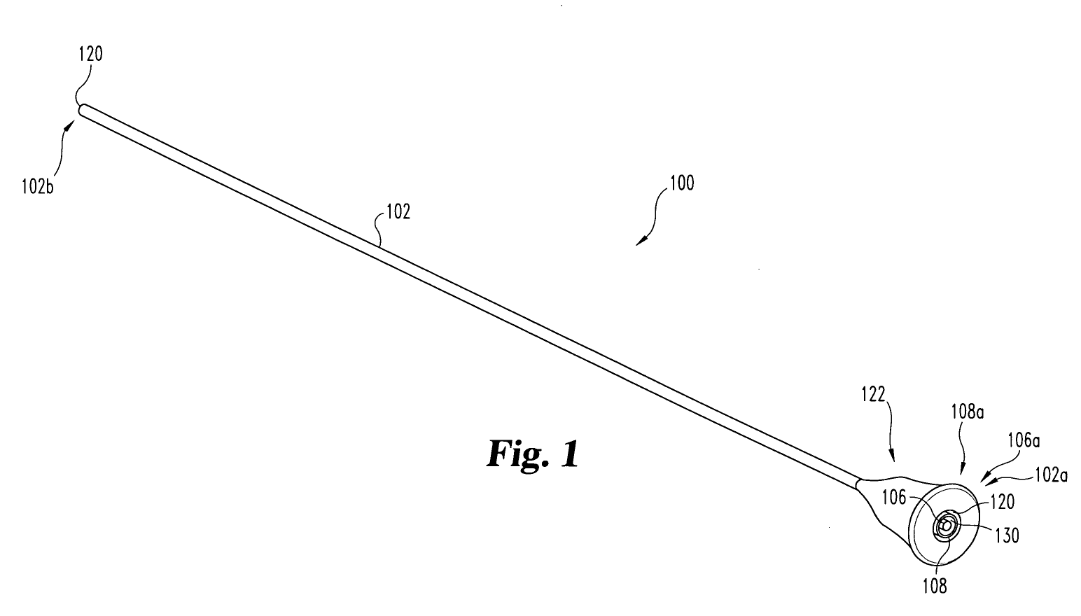

Generally referring to FIGS. 1-8, there is shown an embodiment of a temporary anchorable tether system 100. System 100 is operable to temporarily anchor into tissue or bone, or other parts of a patient's body, via deployment of one or more elastic anchoring members at a desired location. Accordingly, one or more medical devices can be advanced along one or more components and positioned at the desired location through use of system 100. System 100 generally includes an outer sleeve or sheath tube 102 selectively surrounding at least one elastic anchoring member, such as anchor hooks 104 and 105, in engagement with a guide line, such as tether 106. In certain embodiments, system 100 can further include a hollow rod 108 (which may be thought of as a needle insofar as it can be used to thread through instruments or implants), a crimp 110 joining anchor hooks 104 and 105 with tether 106, and a deployment button 112 for deploying anchor hooks 104 and 105 out of outer sleeve 102.

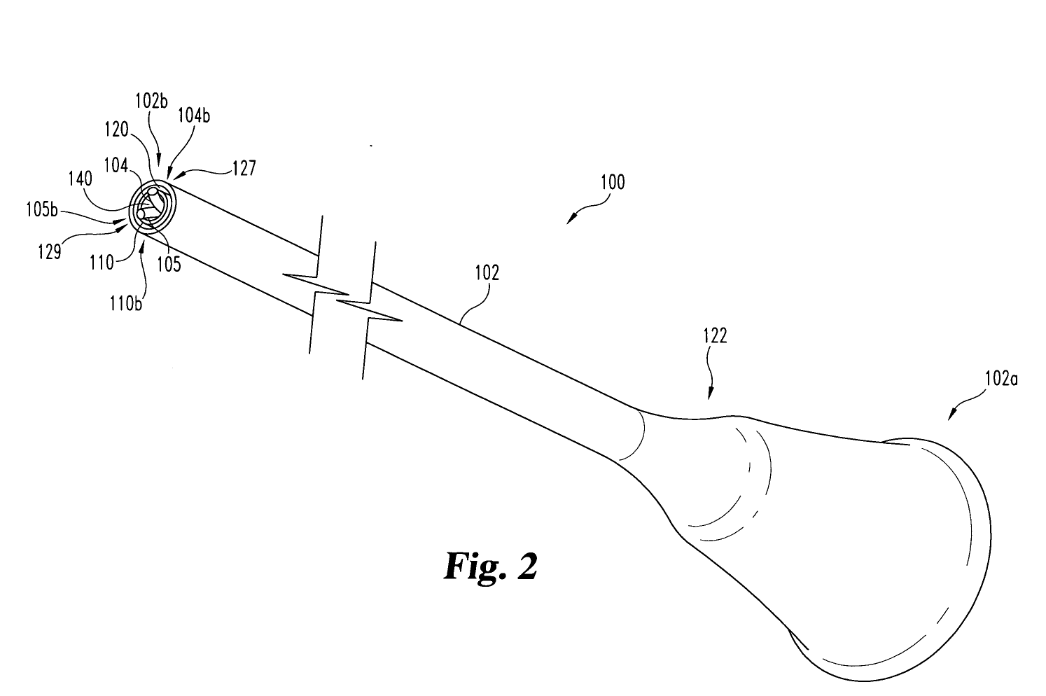

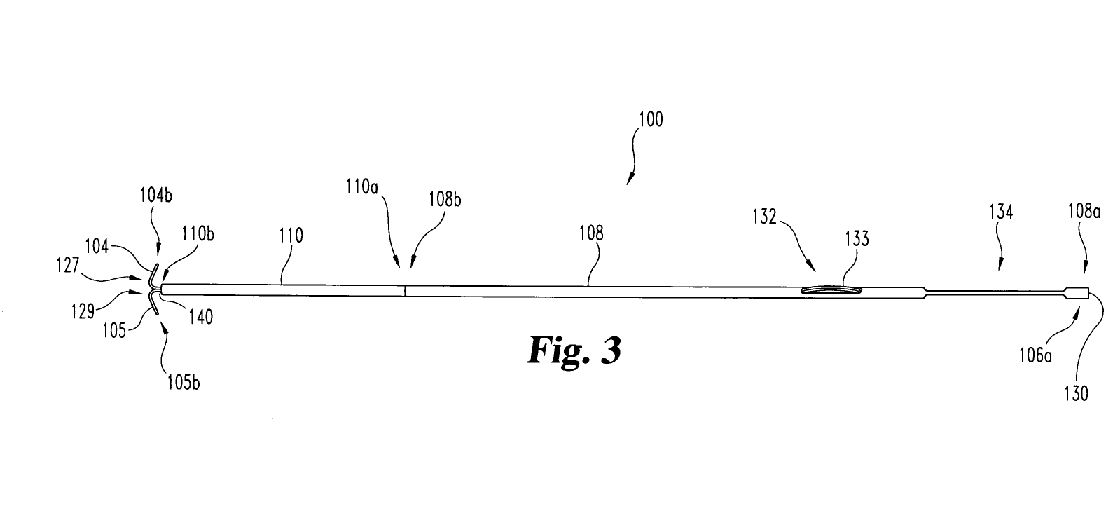

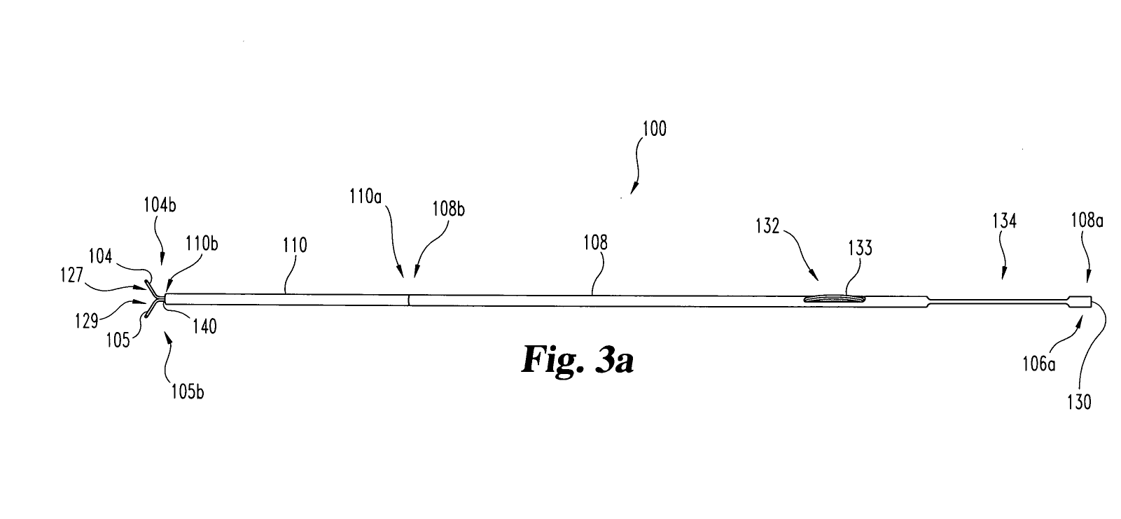

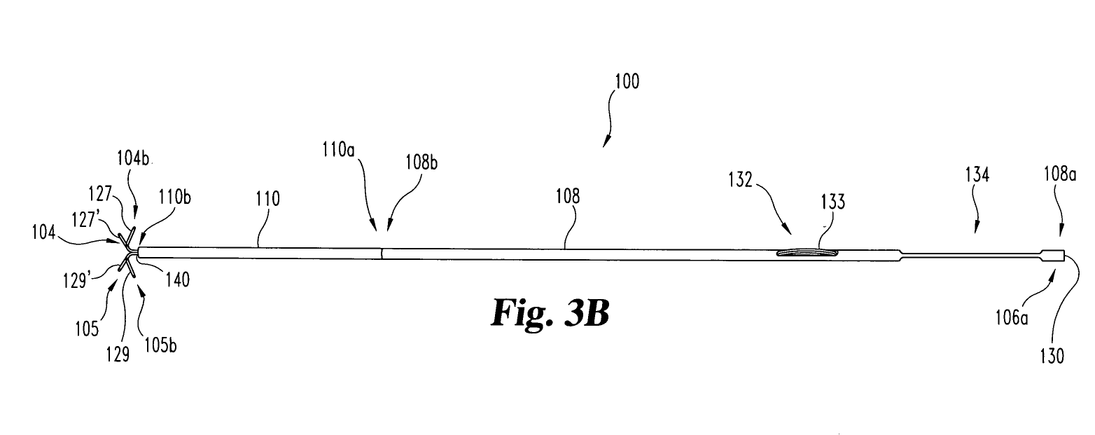

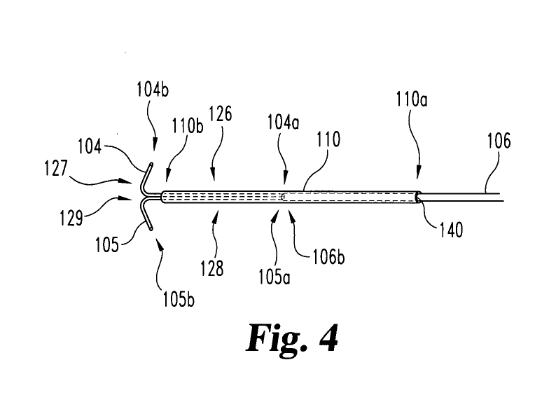

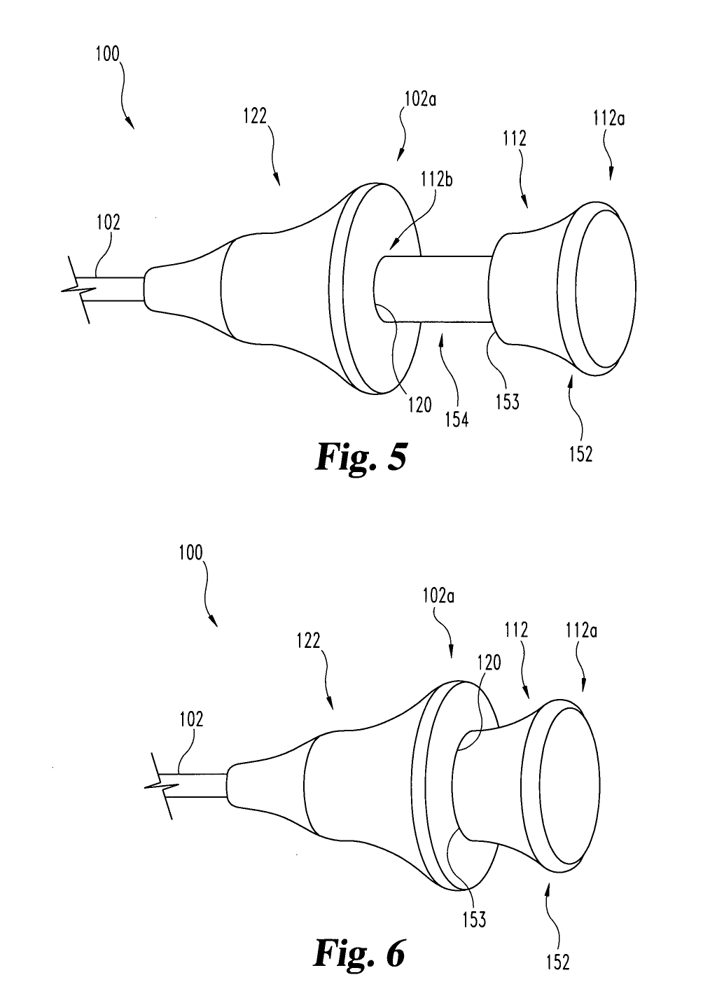

FIGS. 1 and 2 illustrate many of the components of system 100 as assembled. Additionally, FIGS. 3 and 4 shown certain components of system 100, while other components are absent for illustration purposes. Further, FIGS. 5-8 illustrate certain aspects of the methods of using system 100, among other things.

Outer sleeve 102 includes a proximal end 102a and a distal end 102b and defines an elongated bore 120 extending therethrough. In certain embodiments, bore 120 is sized to receive rod 108 having tether 106 disposed therein, crimp 110, and anchor hooks 104 and 105 in a deformed position, as will be explained in greater detail. As illustrated, outer sleeve 102 may optionally include a handle portion 122 adjacent proximal end 102a, with bore 120 extending therethrough. In the illustrated embodiment, handle portion 122 is generally increasing in diameter in a direction toward proximal end 102a. However, it should be appreciated that handle portion 122 (if present) can be configured differently than as illustrated. In certain embodiments, outer sleeve 102 is composed of a substantially rigid material. In certain other embodiments, outer sleeve 102 is composed of a substantially flexible material allowing for sufficient flexibility of outer sleeve 102 during positioning in a patient's body. In the illustrated embodiment, outer sleeve 102 includes an elongated tubular shape; however, it should be appreciated that outer sleeve 102 can be sized and shaped differently.

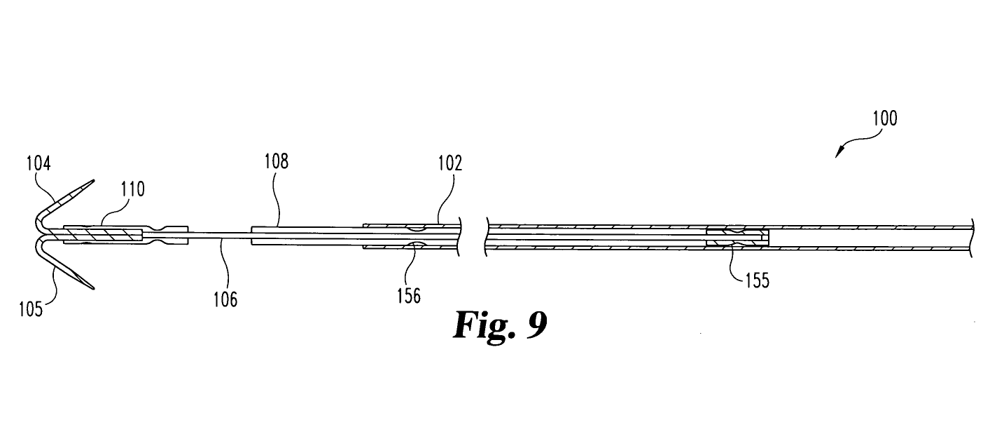

In the illustrated embodiment, there are two anchor hooks 104 and 105 used in conjunction with system 100. In other embodiments, there may be more or less than two elastic anchor members, such as anchor hooks, used with system 100. Additionally, the elastic anchor member(s) used with system 100 can include other appropriate shapes configured for anchoring into bone. In certain embodiments, the configurations of the elastic anchor member(s) reduce the likelihood of damage to surrounding tissue or bone via reducing or eliminating any advancement of the anchor member(s) into the bone or tissue upon use of system 100. Hooks 104 and 105 include proximal ends 104a and 105a and distal ends 104b and 105b, respectively. Additionally, hooks 104 and 105 include elongated portions 126 and 128 adjacent proximal ends 104a and 105a, respectively. Hooks 104 and 105 may further include anchor portions 127 and 129 adjacent distal ends 104b and 105b, respectively. Anchor portions 127 and 129 may have substantially blunt ends (e.g. FIG. 3), or may have relatively sharp or pointed ends (e.g. FIG. 9). As illustrated, anchor hooks 104 and 105 are attached to tether 106 (as best seen in FIG. 4). In certain embodiments, anchor hooks 104 and 105 are welded or otherwise fastened to tether 106. It is contemplated that anchor hooks 104 and 105 may be attached or engaged with tether 106 in other appropriate manners.

In certain embodiments, anchor hooks 104 and 105 are composed of an elastic or pseudoelastic material. Additionally, in certain embodiments, anchor hooks 104 and 105 are composed of a shape memory alloy, such as nickel titanium as an example. It is contemplated that anchor hooks 104 and 105 can be composed of one or more suitable self-expanding materials. Anchor hooks 104 and 105 each include a natural or unstressed curved configuration (see, e.g., FIGS. 3, 3A, 3B, 4). Anchor hooks 104 and 105 may be elastically deformed to an undeployed configuration (e.g. FIG. 2), in which anchor portions 127 and 129 are substantially straightened and confined within bore 120 of outer sleeve 102. Upon deployment from bore 120 of outer sleeve 102, anchor portions 104b and 105b of anchor hooks 104 and 105 reform to their natural curved configurations, to anchor into bone or other tissue. In the illustrated embodiment, outer sleeve 102 and corresponding bore 120 are generally straight and anchor portions 127 and 129 of anchor hooks 104 and 105 are generally curved in their natural or unstressed states. However, it should be appreciated that the configurations of outer sleeve 102 and anchor hooks 104 and 105 are illustrated as examples only and other configurations are contemplated.

In the embodiment shown in FIG. 3, anchor portions 127 and 129 point generally rearward, i.e. generally toward handle 122, when extended from sleeve 102. FIG. 3A shows a different configuration in which anchor portions 127 and 129 point generally forward, i.e. generally away from handle 122, when extended from sleeve 102. Having rearward-pointing anchor portions can help prevent further insertion of system 100. An embodiment in which system 100 includes both rearward-pointing anchor portions 127 and 129 and forward-pointing anchor portions 127′ and 129′ is shown in FIG. 3B. Anchor portions 127 and 127′ are shown as part of one anchor 104, and anchor portions 129 and 129′ are shown as part of anchor 105. In other embodiments, separate anchors could be provided for anchor portions 127, 127′, 129 and 129′.

Tether 106 includes a proximal end 106a and a distal end 106b. In certain embodiments, tether 106 is composed of a suture material; however, it is contemplated that other appropriate substantially flexible materials may be used as tether 106. As provided above, in certain embodiments, distal end 106b of tether 106 is connected or engaged with proximal ends 104a and 105a of anchor hooks 104 and 105. Tether 106 may include a length sufficient to span from a desired location within the patient's body to outside the patient's body. Additionally, tether 106 may be sized to allow for threading of medial devices such as implants or instruments over the tether to position the medical devices at the desired location.

Rod 108 includes a proximal end 108a and a distal end 108b and defines an elongated bore 130. In certain embodiments, bore 130 is sized and configured to slidably receive tether 106 therein. When system 100 is assembled, distal end 108b is positioned adjacent or abutting crimp 110 such that rod 108 is generally aligned with crimp 110 (as best seen in FIG. 3). Additionally, upon assembly, rod 108 is slidably movable within bore 120 of outer sleeve 102. In certain embodiments, bore 130 of rod 108 is concentric with bore 120 of outer sleeve 102. Additionally, proximal end 108a of rod 108 may be substantially aligned with proximal end 102a of outer sleeve 102 (as best seen in FIG. 1). In other embodiments, a portion of rod 108 adjacent proximal end 108a may extend out of bore 120 of outer sleeve 102 and out of handle portion 122.

Rod 108 may include a window 132 defined therein configured to receive an undeployed length of tether material 133 of tether 106. In certain embodiments, material 133 is looped through window 132 as necessary such that proximal end 106a of tether 106 is substantially aligned with proximal end 108a of rod 108 (as best seen in FIG. 3). Additionally, rod 108 may optionally include a crimped section 134 defined therein to secure tether 106 within bore 130 of rod 108. In certain embodiments, upon alignment of proximal end 106a of tether 106 with proximal end 108a of rod 108, an appropriate medical instrument may be used to create crimped section 134 to secure the engagement of tether 106 in bore 130 of rod 108 (as best seen in FIG. 3). Crimped section 134 may substantially prevent tether 106 from disengagement with rod 108. In the illustrated embodiment, crimped section 134 is shown proximate the proximal end 108a. However, it should be appreciated that crimped section 134 can be located at other positions along rod 108. In certain embodiments, crimped section 134 is positioned proximal of window 132. In certain embodiments, rod 108 is composed of a substantially rigid material. As an example, rod 108 may be composed of stainless steel in certain embodiments.

Additionally, system 100 may include crimp 110. Crimp 110 includes a proximal end 110a and a distal end 100b and defines an elongated bore 140 therein. Crimp 110 is configured to join anchors 104 and 105 with tether 106 (see, e.g., FIGS. 4 and 9). In certain embodiments, distal end 110b is positioned adjacent anchor portions 127 and 129 of anchor hooks 104 and 105, with elongated portions 126 and 128 being substantially positioned and received in bore 140 of crimp 110. In certain embodiments, system 100 is assembled such that proximal end 110a is positioned adjacent and abutting distal end 108b of rod 108. In other embodiments, crimp 110 can be engaged and/or connected with rod 108 in other appropriate manners. Upon assembly, crimp 110 is slidably movable within bore 120 of outer sleeve 102. In certain embodiments, bore 140 of crimp 110 is aligned with bore 130 of rod 108 and concentric with bore 120 of outer sleeve 102. In certain embodiments, crimp 110 is composed of a substantially rigid material, such as stainless steel as an example. As seen in FIG. 9, crimp 110 can be crimped at two places to join tether 106 and anchors 104 and 105, one to hold tether 106 mechanically and/or frictionally, and one to hold anchors 104 and 105 mechanically and/or frictionally. In embodiments in which a substantial portion of tether 106 and anchors 104 and 105 overlap, only a single crimped place may be used to mechanically and/or frictionally hold both tether 106 and anchors 104, 105 together.

As best illustrated in FIGS. 5 and 6, deployment button 112 includes a proximal end 112a and a distal end 112b. Additionally, deployment button 112 may include a handle portion 152 adjacent proximal end 112a and an elongated portion 154 adjacent distal end 112b. In certain embodiments, handle portion 152 includes a larger diameter than elongated portion 154, thereby creating a ledge 153 defined between portions 152 and 154. Distal end 112 may be configured to engage proximal end 108a of rod 108. In certain embodiments, distal end 112b abuts proximal end 108a of rod 108. However, it is contemplated that deployment button 112 can engage or connect with rod 108 in other appropriate manners. Additionally, in certain embodiments, elongated portion 154 may define a blind hole therein to receive a portion of rod 108 adjacent proximal end 108a. As illustrated, elongated portion 154 is sized and configured to be received in bore 120 of outer sleeve 102 (as best seen in FIGS. 5 and 6) to exert a pushing force on rod 108, which in turn pushes crimp 110 to deploy anchor hooks 104 and 105 out of bore 120. In certain embodiments, elongated portion 154 includes a diameter substantially the same as or slightly smaller than the diameter of bore 120 of outer sleeve 102. It should be appreciated that deployment button 112 is only one illustrated example of a deployment mechanism which could be utilized with system 100 and that other deployment mechanisms could be used to deploy anchor portions 127 and 129 of anchor hooks 104 and 105 into bone.

System 100 may also include a tether stop 155 (FIG. 9) crimped or otherwise fastened to an end portion of tether 106 distal from anchors 104, 105, and a second crimp 156. Crimp 156 may be a deformed portion of rod 108, or may be a separate sleeve or other piece in or on rod 108 that is crimped, thereby deforming rod (or needle) 108. Crimp 156 is only fastened to hollow rod 108 in this embodiment, so that movement of tether 106 through rod 108 is substantially or completely unrestricted. Tether stop 155 is sized at least slightly smaller than an inner diameter of rod 108 so that withdrawal of rod 108, as further described below, will not pull out tether stop 155 and tether 106. Tether stop 155 is sized at least slightly larger than an inner diameter of rod 180 where it is crimped by crimp 156, so that rod 108 cannot be removed from around tether 106. In other words, when rod 108 is withdrawn to a degree that the narrowed portion of crimp 156 and rod 108 abuts tether stop 155, further withdrawal of rod 108 is impossible without also pulling tether 106. By preventing rod 108 and tether 106 from being separated from each other, a more rigid and easily-accessed portion of guidewire to pass through any necessary instruments, tools or implants is provided.

The assembly of system 100 will be discussed generally with reference to FIGS. 1-8. Initially, proximal ends 104a and 105a of anchor hooks 104 and 105 may be connected with distal end 106b of tether 106. As stated above, in certain embodiments, proximal ends 104a and 105a are welded to distal end 106b of tether 106; however, it is contemplated that other appropriate manners of connecting anchor hooks 104 and 105 with tether 106 can be used with system 100. Anchor portions 127 and 129 of anchor hooks 104 and 105 assume a natural or unstressed curved configuration (as best seen in FIG. 4). Tether 106 may be inserted through bore 140 of crimp 110 (if used). In certain embodiments, proximal end 106a of tether 106 is inserted through distal end 110b of crimp 110. Crimp 110 may be advanced over tether 106 and elongated portions 126 and 128 of anchor hooks 104 and 106, such that distal end 110b of crimp 110 is positioned adjacent anchor portions 127 and 129 of anchor hooks 104 and 105.

Additionally, tether 106 may be inserted through bore 130 of rod 108. In certain embodiments, proximal end 106a of tether 106 is inserted through distal end 108b of rod 108. Rod 108 may be advanced over tether 106 to a desired position and/or until distal end 108b of rod 108 is positioned adjacent and abutting proximal end 110a of crimp 110 (as best seen in FIG. 3). In certain embodiments, undeployed length of material 133 of tether 106 can be accessed through window 132 and looped therethrough (as best seen in FIG. 3). It is contemplated that material 133 may be looped through window 132 such that proximal end 106a of tether 106 is substantially aligned with proximal end 108a of rod 108. In certain embodiments, a device may be used to crimp rod 108 creating crimped section 134 to secure tether 106 within bore 130 of rod 108 at section 134 (as best seen in FIG. 3). Crimped section 134 substantially maintains engagement between tether 106 and rod 108 such that tether 106 is not slidable within bore 130 at section 134.

Outer sleeve 102 can be engaged with system 100 by inserting proximal end 108a of rod 108 through distal end 102b of outer sleeve 102. Outer sleeve 102 may be advanced over rod 108, crimp 110 and further advanced over anchor hooks 104 and 105 such that anchor portions 127 and 129 elastically deform within bore 120 of outer sleeve 102 (as best seen in FIG. 2). In certain embodiments, anchor portions 127 and 129 elastically deform to substantially straightened, undeployed configurations confined within bore 120 of outer sleeve 102 upon advancement of sleeve 102 over anchors 104 and 105 (as best seen in FIG. 2). Additionally, in certain embodiments, anchor portions 127 and 129 may remain slightly curved within bore 120. Additionally, in certain other embodiments, anchor portions 127 and 129 may deform to completely straight segments. It is contemplated that anchor portions 127 and 129 could elastically deform to various configurations such that anchor portions 127 and 129 are confined within bore 120 of outer sleeve 102.

Generally referring to FIGS. 1-8, the operation and use of system 100 will be described with reference to a surgical procedure involving a section of spine of a patient. It should be appreciated that other uses of system 100 described herein and other surgical procedures can be made.

To treat the condition or injury of the patient, the surgeon obtains access to the surgical site in any appropriate manner, e.g. through incision and retraction of tissues. It is contemplated that system 100 discussed herein can be used in minimally-invasive surgical techniques where the spinal segment is accessed through a micro-incision, a sleeve, or one or more retractors that provide a protected passageway to the area. System 100 discussed herein also has application in open surgical techniques where skin and tissue are incised and retracted to expose the surgical site.

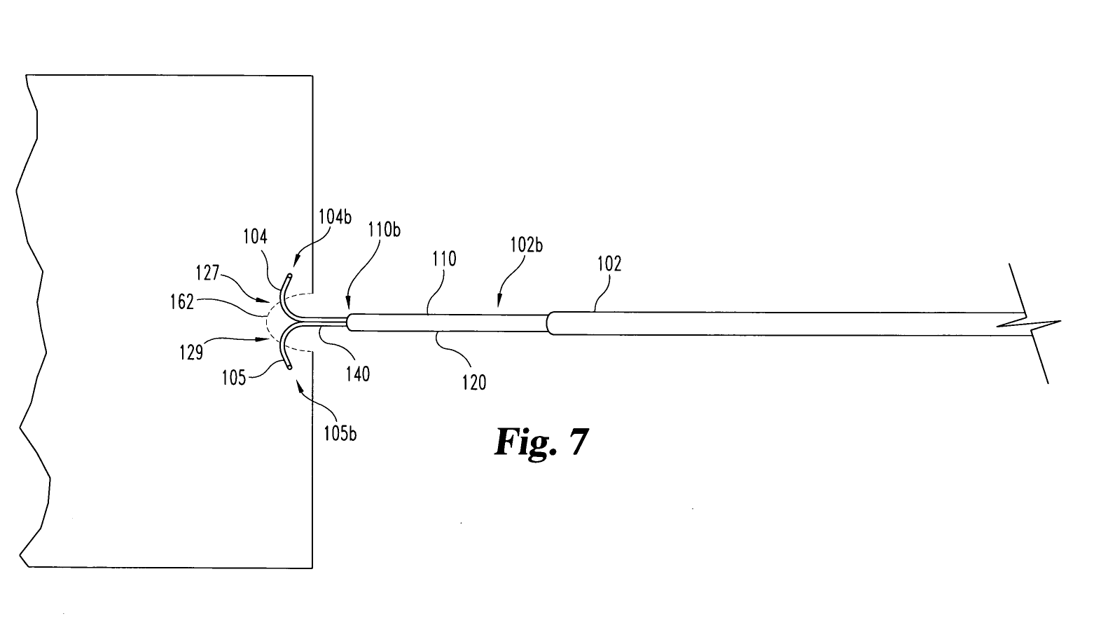

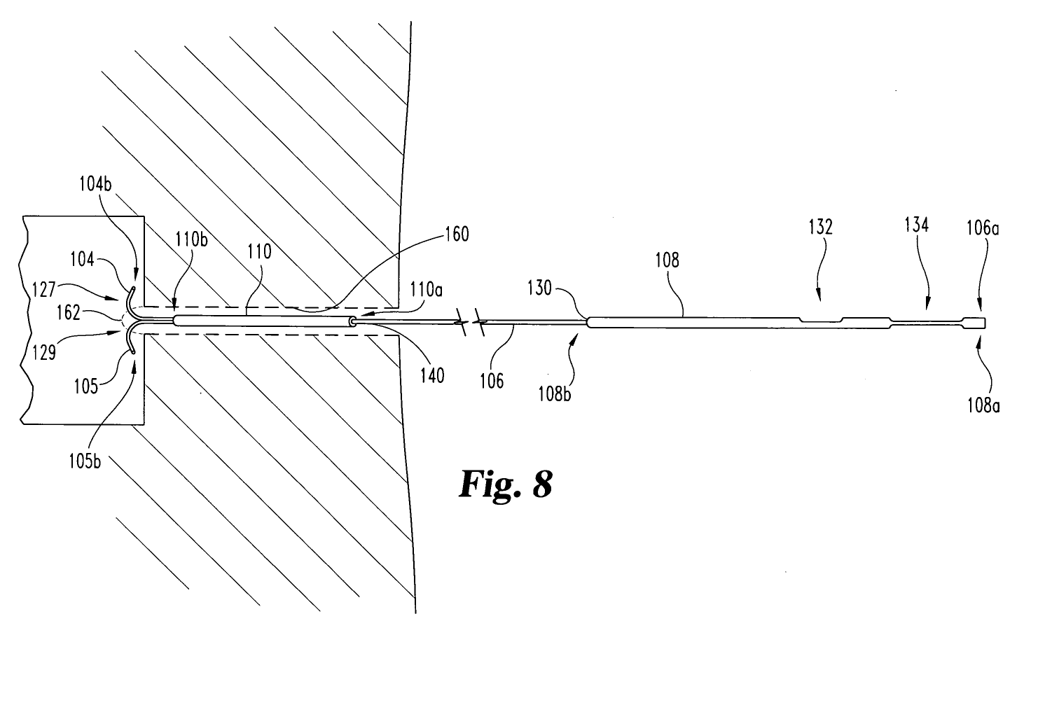

Upon assembly of system 100 as described above, or in other manners as appropriate, system 100 may be used as or similar to a guidewire medical device to provide for the proper advancement and positioning of medical instruments or implants to a desired location within a patient's body. A surgical opening 160 may be made in the patient's body to allow for the insertion and use of system 100 (as best seen in FIG. 8). Additionally, surgical opening 160 may extend from outside the patient's body to the desired location within the patient's body. In certain embodiments, prior to deployment of system 100, a hole 162 may optionally be created in the bone at the desired location (as best seen in FIG. 7). In such embodiments, anchor hooks 104 and 105 can be deployed into hole 162 and at least partially anchored into the surrounding bone or tissue (as best seen in FIG. 7). In certain other embodiments, anchor hooks 104 and 105 are anchored into bone or tissue without a hole being created therein.

System 100 may be deployed causing anchor hooks 104 and 105 to substantially anchor into bone at the desired location. In other embodiments, anchor hooks 104 and 105 are anchored into tissue or other parts of the patient's body. In the illustrated embodiment, anchor hooks 104 and 105 are deployed out of bore 120 of outer sleeve 102 via deployment button 112. However, it should be appreciated that other deployment mechanisms may be used with system 100. In the illustrated embodiment, distal end 112b of deployment button 112 contacts proximal end 108a of rod 108. Additionally, elongated portion 154 of deployment button 112 is inserted into bore 120 at proximal end 102a of outer sleeve 102 and advanced to a position where ledge 153 contacts proximal end 102a of outer sleeve 102 (as best seen in FIGS. 5 and 6). In such embodiments, portion 154 may be advanced through bore 120 at a relatively quick speed. Additionally, in such embodiments, ledge 153 may prohibit further advancement of button 112 within bore 120. Accordingly, button 112 exerts a deployment force on rod 108, which exerts a force on crimp 110, thereby exerting a force on anchor hooks 104 and 105 to expel or deploy the anchor hooks from bore 120 and anchor the hooks into bone or tissue at the desired location. Upon deployment, anchor portions 127 and 129 of anchor hooks 104 and 105 may return toward their first natural or unstressed curved configurations (as best seen in FIG. 7) to better enable anchoring into the bone. In certain embodiments, the curved configurations of anchor portions 127 and 129 may be thought of as deployed configurations in which anchor portions 127 and 129 at least partially anchor into bone at the desired location.

Upon deployment of system 100 and anchoring of hooks 104 and 105 at the desired position, outer sleeve 102 may be retracted from rod 108 (as best seen in FIG. 8). In certain embodiments, elongated portion 154 of button 112 creates a friction fit inside bore 120 and can also be removed with outer sleeve 102. Deployment of system 100 and anchoring of hooks 104 and 105 into bone may cause the retraction of undeployed material 133 of tether 106 from window 132 of rod 108. Additionally or alternatively, rod 108 may be pulled in a direction away from anchor hooks 104 and 105 to retract material 133 from window 132. In certain embodiments, rod 108 may be pulled to a position outside of the patient's body. Retraction of material 133 out window 132 extends the distance between anchor hooks 104 and 105 and proximal end 108a of rod 108 and also separates rod 108 from crimp 110. In certain embodiments, crimped section 134 maintains the engagement between tether 106 and rod 108 adjacent proximal end 108a.

Once access to the surgical site has been obtained and system 100 has been properly deployed and anchored, the surgeon may advance one or more medical devices to a position adjacent the bone, such as vertebrae of a spinal segment that require compression, distraction and/or support in order to relieve or improve their condition. Medical devices such as instruments or implants may be advanced along rod 108, tether 106 and crimp 110 by initially threading proximal end 108a of rod 108 through a cannulation or other such lumen or hole in a selected instrument or implant. In certain embodiments, the outer diameters of the rod 108, tether 106 and crimp 110 are selected and designed in relation to the lumens or holes defined the various medical devices to be used in connection with system 100, so that the medical devices can be advanced over the components as described herein. The selected medical device can then be positioned adjacent anchor portions 127 and 129 of hooks 104 and 105 at the desired location in the patient's body for the necessary medical application. In such embodiments, the configurations of anchor portions 127 and 129 and the use of system 100 may be thought of as a third guidewire configuration, in which one or more medical devices may be advanced to the desired location in the patient's body.

Following use, anchor hooks 104 and 105 may be pulled or withdrawn through the medical device (e.g. cannulated bone screw) used in connection with system 100. Accordingly, anchor portions 127 and 129 elastically deform into the cannulation or lumen of the medical device. In certain embodiments, anchor portions 127 and 129 may reform to their natural curved configurations upon exiting the cannulation of the medical device. In certain other embodiments, anchor portions 127 and 129 may remain anchored into bone or tissue at the desired location. It is contemplated that system 100 may be designed and configured for repeated use with various medical devices. Additionally, in other cases, it is contemplated that system 100 is designed and configured for a single use.

As an example, the medical devices used with system 100 may be bone implant members, such as bone screws. The bone screws may be advanced to the desired accessed location via system 100. In such cases, pilot holes in vertebrae may be made and threaded bone-engaging portions of the screws may be inserted into or otherwise connected to a vertebral body. Bone engaging portions of the screws can be threaded into the vertebrae to a desired depth and/or desired orientation relative to a longitudinal axis of the spinal segment. In certain embodiments, the surgeon or other medical professional can use a driving tool or other similar instrument to advance the screws.

Thus, in general system 100 can deliver and deploy a guidewire for a bone screw or other implants or tools, the guidewire having a flexible and/or shape memory (e.g. Nitinol) anchor to provide a counter traction, preventing the guide wire from being pulled out while being manipulated. In one example, after a hollow drill has bored a hole into bone, the device is placed through the drill bit and into the drilled hole. When the drill bit is removed, sheath tube or sleeve 102 is retracted while push rod 108 is held in place, deploying anchor hooks 104 and 105. Next sleeve 102 is removed completely. Rod 108 is then retracted as well, exposing tether 106 and providing a flexible portion of guide wire (in tether 106) that may be set aside. Crimp 156 in rod 108 along with tether stop 155, in embodiments in which they are provided, prevent rod 108 and tether 106 from being separated from each other, providing a more rigid portion of guide wire to pass through any necessary instruments, tools or implants.

In certain embodiments, one or more of outer sleeve 102, rod 108, crimp 110, and button 112 are composed of biocompatible, metallic materials. However, it should be appreciated that any or all of outer sleeve 102, rod 108, crimp 110, and button 112 can be formed with one or more of a variety of materials. These materials may be rigid, malleable, semi-flexible, or flexible. The material(s) selected for a particular component of system 100 can depend on a number of factors including but not limited to the intended use of the system, as well as its size, shape, and configuration. In general, suitable material(s) will be selected to allow for a certain desired performance and other characteristics, for example, to exhibit a flexibility falling within a desired range and/or to have shape memory, as with anchor hooks 104 and 105.

Suitable biocompatible metallic materials that can be used in one or more components of system 100 include but are not limited to gold, rhenium, platinum, palladium, rhodium, ruthenium, various stainless steels, tungsten, titanium, nickel, cobalt, tantalum, iron, and copper, as well as alloys of these and other suitable metals, e.g., cobalt alloys, a cobalt-chromium-nickel alloy, a nickel-cobalt-chromium-molybdenum alloy, and a nickel-titanium alloy. In certain aspects, an alloy is selected that exhibits desired biocompatibility and includes suitable strength and ductility to perform in accordance with the methods described herein. In certain embodiments, synthetic polymeric materials, including bioresorbable and/or non-bioresorbable plastics may be used to form one or more components of system 100. Further, one or more suitable ceramic materials may be used to form one or more components of system 100. Moreover, it is contemplated that one or more components of system 100 may include a suitable biocompatible coating thereon.

While the disclosure has been illustrated and described in detail in the drawings and foregoing description, the same is to be considered as illustrative and not restrictive in character. It should be understood that only certain embodiments have been shown and described and that all changes and modifications that come within the spirit of the disclosure are desired to be protected.

What is claimed is:

1. A surgical apparatus for introducing a medical device through a surgical opening in a body and guiding the position of the device to a desired location, comprising:
an outer sleeve having a proximal end and a distal end and defining an elongated bore;
a tether having a proximal end and a distal end and being slidably moveable within said bore; and
at least one elastic anchor hook engaged with said distal end of said tether, wherein said anchor hook is configured to at least partially anchor into bone at the desired location;
wherein said anchor hook is elastically deformable from an unstressed first shape being substantially curved to a deformed second shape being substantially straightened, wherein said anchor hook is configured to deform to said second shape when positioned substantially within said bore of said outer sleeve and substantially resume to said unstressed first shape upon deployment from said bore of said outer sleeve.

2. The apparatus of claim 1, wherein said anchor hook is at least partially composed of a pseudoelastic material.

3. The apparatus of claim 1, wherein said at least one anchor hook includes two mirrored anchor hooks positioned in substantially the same plane.

4. The apparatus of claim 3, wherein said anchor hooks have end portions, and when said anchor hooks have said unstressed first shape, said end portions point substantially rearward relative to said sleeve and said tether.

5. The apparatus of claim 4, wherein when said anchor hooks have said unstressed first shape, at least one of said end portions points substantially rearward relative to said sleeve and said tether, and at least one of said end portions points substantially forward relative to said sleeve and said tether.

6. The apparatus of claim 3, wherein said anchor hooks have end portions, and when said anchor hooks have said unstressed first shape, said end portions point substantially forward relative to said sleeve and said tether.

7. The apparatus of claim 1, comprising a crimp partially surrounding said tether and said anchor hooks and being sized to be received in said bore of said outer sleeve.

8. The apparatus of claim 7, wherein said crimp is composed of stainless steel.

9. The apparatus of claim 1, wherein said anchor hook is composed of a shape memory alloy.

10. The apparatus of claim 9, wherein said shape memory alloy is a nickel titanium alloy.

11. The apparatus of claim 1, comprising a rod having a proximal end and a distal end and defining an elongated bore, wherein said rod is configured to receive said tether in said bore of said rod.

12. The apparatus of claim 11, further comprising a crimp attached to said proximal end of said tether and a crimp in or on said rod, wherein said rod crimp cannot pass said tether crimp.

13. The apparatus of claim 11, wherein said rod includes at least one window and a portion of said tether is looped through said window such that said portion extends at least partially out said window, wherein said portion of said tether is configured to be retracted through said window upon deployment of said anchor hook out said outer sleeve.

14. The apparatus of claim 11, comprising a deployment button configured to engage said rod, wherein said deployment button is operable to expel said anchor hook out of said bore of said outer sleeve to anchor said anchor hook at least partially into bone, and wherein said deployment button includes an extension configured to be received and travel in said bore of said outer sleeve.

15. The apparatus of claim 11, wherein said rod includes a crimped section of reduced diameter configured to join together said tether and said rod.

16. The apparatus of claim 15, wherein said crimped section is proximate said proximal end of said rod.

17. The apparatus of claim 1, wherein the apparatus includes:
a first undeployed configuration, wherein said undeployed configuration includes said tether and said anchor hook being received in said bore of said outer sleeve, with said anchor hook including said deformed second shape;
a second deployed configuration, wherein said second deployed configuration includes a portion of said tether received in said bore of said outer sleeve and said anchor hook at least partially anchored into bone at a desired location, with said anchor hook including said unstressed first shape; and
a third guidewire configuration, wherein said third guidewire configuration includes said outer sleeve removed from said tether to allow for at least one medical device to be inserted over said tether and positioned adjacent said anchor hook at the desired location.

18. A surgical apparatus for introducing a medical device through a surgical opening in a body and guiding the position of the device to a desired location, comprising:
an outer sleeve having a proximal end and a distal end and defining an elongated bore;
a tether member having a proximal end and a distal end and being slidably moveable within said bore;
at least one elastic anchor member engaged with said distal end of said tether and configured to at least partially anchor into bone, wherein said anchor member is composed of a shape memory alloy and is configurable in at least two configurations including an unstressed curved configuration and a deformed configuration;
a first undeployed configuration wherein said tether and said anchor hook are received in said bore of said outer sleeve, wherein said first undeployed configuration includes said anchor member configured in said deformed configuration, said anchor member being constrained by said bore of said outer sleeve;
a second deployed configuration wherein said anchor member is deployed from said bore of said outer sleeve and at least partially anchors into bone, wherein said second deployed configuration includes said anchor member reforming toward said curved configuration; and
a third guidewire configuration wherein said outer sleeve is removed from said tether to allow for at least one medical device to be inserted over said tether and positioned adjacent said anchor member.

19. The apparatus of claim 18, wherein said at least one anchor member includes two mirrored anchor hooks.

20. The apparatus of claim 18, comprising a crimp defining an elongated bore for receiving part of said tether and said anchor member, said crimp being sized to be received in said bore of said outer sleeve.

21. The apparatus of claim 18, wherein said shape memory alloy is a nickel titanium alloy.

22. The apparatus of claim 18, comprising a rod having a proximal end and a distal end and defining an elongated bore, wherein at least a portion of said tether is received in said bore of said rod and said distal end of said rod is positionable adjacent said proximal end of said crimp, wherein said rod is configured to be received in said bore of said outer sleeve.

23. The apparatus of claim 22, comprising a deployment button configured to engage said rod and configured to be partially received in said bore of said outer sleeve, wherein said deployment button is operable to expel said anchor member out of said bore of said outer sleeve and at least partially anchor into bone according to said second deployed configuration.

24. A method, comprising:
providing a guidewire device including a tether having a distal end and a proximal end and at least one elastic anchor hook adjacent said distal end of said tether, wherein said anchor hook is composed of a shape memory material and includes a first unstressed position and a second deformed position, said first position being substantially curved and said second position being substantially straightened;
providing a rod having a distal end and a proximal end and defining an elongated bore sized for receipt of said tether;
inserting at least part of said tether through said bore of said rod;
providing an outer sleeve having a distal end and a proximal end and defining an elongated bore sized for receipt of said rod and said anchor hook in said second deformed position;
inserting said outer sleeve over said rod and said anchor hook; and
deforming said anchor hook to said second deformed position within said bore of said outer sleeve.

25. The method of claim 24, comprising providing a crimp defining an elongated bore sized to receive part of said tether and said anchor hook and positioning said crimp around part of said tether and said anchor hook.

26. The method of claim 24, comprising providing a deployment button, said button being selectively moveable within said bore of said outer sleeve to deploy said anchor hook out of said bore of said outer sleeve and into bone.

27. The method of claim 24, wherein said rod includes a window defined therein, the method comprising looping an excess portion of said tether through said window.

28. A method, comprising:
providing a guidewire device including a tether having a distal end and a proximal end and at least one elastic anchor hook adjacent said distal end of said tether, wherein said anchor hook is composed of a shape memory material and is configurable in a first unstressed position and a second deformed position, wherein said guidewire device further includes a retractable outer sleeve having a distal end and a proximal end and defining an elongated bore, wherein said anchor hook and said tether are positioned in said bore of said outer sleeve with said anchor hook being deformed to said second deformed position within said bore of said outer sleeve;
deploying said anchor hook out of said bore of said outer sleeve, wherein said anchor hook reforms toward said first unstressed position;
anchoring said anchor hook into bone at a desired location;
removing said outer sleeve from surrounding said tether; and
advancing a medical device over said tether to position said device adjacent said anchor hook at said desired location, wherein said medical device includes a cannulation for passage of said tether.

29. The method of claim 28, wherein said first position is substantially curved and said second position is substantially straightened.

30. The method of claim 28, wherein said device further includes a rod having a distal end and a proximal end and defining an elongated bore, wherein at least a portion of said tether is received in said bore of said rod and said rod is configured to be received in said bore of said outer sleeve.

31. The method of claim 30, comprising crimping a section of said rod to join said tether and said rod.

32. The method of claim 30, wherein said rod includes a window defined therein, the method comprising looping an excess portion of said tether through said window.

33. The method of claim 32, comprising pulling said rod in a direction away from said anchor hook to retract said excess portion of said tether out said window.

34. The method of claim 28, comprising providing a crimp defining an elongated bore, wherein part of said tether and said anchor hook are received in said bore of said crimp.

35. The method of claim 28, comprising providing a deployment button, said button being selectively moveable within said bore of said outer sleeve to deploy said anchor hook out of said bore of said outer sleeve and into bone.

36. The method of claim 28, comprising drilling a hole in bone at said desired location, wherein said anchor hook is deployed into said hole in bone.

37. The method of claim 28, comprising removing said tether and said anchor hook from said desired location, wherein said removing comprises withdrawing said anchor hook through said cannulation in said medical device such that anchor hook elastically deforms to said second deformed position.

38. The method of claim 28, wherein said shape memory material is a shape memory alloy.
